(19) United States
(12) Patent Application Publication
(10) Pub. No.: US 2026/0083340 A1
(43) Pub. Date: Mar. 26, 2026

(54) Smart Ring Devices and Systems for Physical and Mental Health Monitoring and Services

(71) Applicant: NANJING NINGKANG ZHONGKE MEDICAL TECHNOLOGIES LTD., Nanjing (CN)

(72) Inventors: Jiankang WU, Nanjing (CN); Zhixi YAO, Nanjing (CN); Xiangtao YAO, Nanjing (CN)

(73) Assignee: NANJING NINGKANG ZHONGKE MEDICAL TECHNOLOGIES LTD., Nanjing (CN)

(21) Appl. No.: 19/409,544

(22) Filed: Dec. 4, 2025

Related U.S. Application Data
(63) Continuation of application No. PCT/CN2023/089408, filed on Apr. 20, 2023.

(51) Int. Cl. A61B 5/024; A61B 5/00; A61B 5/0205; A61B 5/08; A63B 23/18; A63B 24/00; G16H 30/40

(52) U.S. Cl. CPC A61B 5/02405; A61B 5/0205; A61B 5/6826; A61B 5/725; A61B 5/7257; A61B 5/7278; A63B 23/18; A63B 24/0075; G16H 30/40; A61B 5/02416; A61B 5/02438; A61B 5/0816; A61B 5/4041; A61B 5/4884

(57) ABSTRACT

The present invention relates to a system and method for physical and mental health monitoring and services. The system comprises: at least one miniature wearable vital sign signal acquisition device, which acquires and/or calculates at least the following data of a user: heart rate, movement, blood oxygen, and PPG, and sends the acquired and/or calculated data to a user APP in the form of raw data and/or processed data; a user APP, which analyzes the received data; and a data server (300), which is capable of communicating with the user APP to provide data services. According to the present invention, the system guides the user to perform specific tests through the miniature wearable vital sign signal acquisition device and/or the user APP, and performs multi-vital-sign data integrative analysis based on the data acquired and/or calculated during these specific tests through the user APP.

### BACKGROUND OF THE INVENTION

### 1. Technical Field

The present invention pertains to the field of medical health monitoring and intervention technology, specifically to smart ring devices and systems for physical and mental health monitoring and services.

### 2. Background Technology

With the development of microelectronics and Artificial Intelligence, it has become possible to monitor health and enjoy intelligent services anytime, anywhere, and on-the-go. The smart ring, due to its convenient wearability and reliability in acquiring human vital signs, together with smartphone APPs, constitutes the most widely accepted medical and health equipment and service system that are currently emerging.

Existing technologies related to smart rings include, for example:
The Chinese patent application titled “A Smart Ring Capable of Health Monitoring”, with application No. 202210318875.0, established a wireless connection between the smart ring and a mobile terminal device, enabling device management, real-time data acquisition, control, storage, historical data statistics, health analysis, and other operations through an APP. It features functions such as step counting, calorie consumption, heart rate, blood oxygen, body temperature, HRV, respiratory rate, sleep monitoring, NFC, and wireless charging;
The US patent application titled “Sensing System and Method for Smart Ring Employing Sensor Spatial Diversity”, with application number U.S. Ser. No. 17/810,405, discloses a smart ring that acquires PPG (photoelectric plethysmography) signals in real time, which is applied to user identification.

In addition, Apple® utilizes the smart ring as an interactive tool.

In addition, in terms of health monitoring smart rings, existing technologies include, for example:
The US patent application titled “an Optical Sensor System of a Wearable Device, a Method for Controlling the Operation of an Optical Sensor System, and a Corresponding Computer Program Product” with publication number US2021204815A1, relates to the use of light emitting and light receiving sensors and a microprocessor in the smart ring itself to monitor and process PPG signal circuits;
The US patent titled “Method for Monitoring Activity of Subject and Monitoring Device Therefor”, with patent number U.S. Pat. No. 10,834,535, relates to the use of an acceleration sensor to monitor activity;
The US patent titled “System and Method for Indicating Information Representing Battery Status of an Electronic Device”, with patent number U.S. Pat. No. 11,075,538 relates to detection of battery charge and protection circuits.

However, with the advancements in technology, particularly in application-specific chip technology, technologies such as using optical sensors to monitor and process PPG to obtain heart rate or heartbeat intervals, using optical sensors with different spectra to measure blood oxygen, using three-dimensional accelerometers to monitor activity, and detecting and managing battery power have been integrated into chips and become publicly available market products.

The US patent titled “Wearable Electronic Device and Method for Manufacturing Thereof”, with U.S. Pat. No. 10,893,833, discloses a finger ring that encapsulates a circuit board with inner and outer layers made of non-ceramic materials, enabling the acquisition of vital signs. The same technology and process are also present in the aforementioned Chinese patent application 202210318875.0 and US patent application U.S. Ser. No. 17/810,405.

In addition, the application of smart rings in healthcare is also related to smartphone APPs and the services provided by servers. Relevant existing technologies include, for example:
The US patent titled “Method and System for Monitoring and Improving Sleep Pattern of User, with patent number U.S. Pat. No. 11,478,187, discloses a method for determining sleep scores based on acquired information sets, measurement data sets, circadian rhythms, and the user's sleep duration. By analyzing the user's sleep scores and related bedtime or fall asleep time, the optimal bedtime window for the user is determined, and feedback is provided to the user;
The US patent titled “Method and System for Assessing a Readiness Score of a User, with patent number U.S. Pat. No. 10,842,429, discloses a method and system for assessing user readiness. This readiness score integrates activity scores during activity periods and sleep scores during rest periods. The readiness score indicates whether the user can fully engage in a certain task or activity;
The US patent titled “Method and System for Providing Feedback to User for Improving Performance Level Management Thereof, with publication number US2021007658A1, evaluates user performance;
The US patent “System and Method for Guiding a User to Improve General Performance”, with publication number US2021241649A1, aims to enhance user performance. It discloses a method for guiding users to sleep and engage in intended activities during optimal time periods based on their sleep and activity patterns throughout the day;
The US patent titled “Method and System for Defining Balance between Physical Activity and Rest, with patent number U.S. Pat. No. 10,105,095, discloses a method for defining the balance between physical activity and rest for users. Based on sensor data, multiple activity-rest parameters are calculated to obtain an activity-rest score, thus providing a feedback reflecting the balance state between activity and rest.

In the prior art of smart ring of health monitoring, achievements have been made in utilizing sleep scoring or activity scoring, evaluating energy and performance, discovering personal rest and activity patterns, and providing feedback. However, there is a lack of integrated analysis for the acquired PPG signals, activity, and body temperature data. As a result, indicators and parameters with vital signs and clinical application value have not been derived, and there is also a lack of professional applications for physical and mental health monitoring and services.

### SUMMARY OF THE INVENTION

In light of the aforementioned prior art, the present invention proposes a smart ring device and system for physical and mental health monitoring and services.

The first aspect of the present invention provides a system for physical and mental health monitoring and services, comprising: at least one miniature wearable vital sign signal acquisition device, which is used to acquire and/or calculate at least the following data of a user: heart rate, movement, blood oxygen and PPG, and send the acquired and/or calculated data to a user APP in the form of raw data and/or processed data; a user APP, which is used to analyze the received data; and a data server, which is used to communicate with the user APP to provide data services; wherein the system is used to guide the user to perform specific tests through the at least one miniature wearable vital sign signal acquisition device and/or the user APP, and perform multi-vital-sign data integrative analysis based on the data acquired and/or calculated during the specific tests through the user APP.

Therefore, according to the present invention, an innovative integrative analysis technology for multi-vital-sign information is provided, offering users professional monitoring and services for physical and mental health.

In one embodiment, the multi-vital-sign data integrative analysis is based on the established physiological mathematical model of autonomic nervous heart rate regulation that integrates multi-vital-sign signals, represented by the following formula:

$${M}_{s} = {A}_{s} {f}_{s} ( {n}_{act} , {n}_{respir} , {n}_{stress} , {n}_{infl} , {n}_{others} ) \qquad ( 1 )$$

$${M}_{p} = {A}_{p} {f}_{p} ( {n}_{act} , {n}_{respir} , {n}_{stress} , {n}_{infl} , {n}_{others} ) \qquad ( 2 )$$

$$H R = H {R}_{Intrinsic} ( 1 + {M}_{S} {C}_{NE} + {M}_{P} {C}_{ACh} ) \qquad ( 3 )$$

Wherein, Mₛ and Mₚ represents the neurotransmitters of the sympathetic and parasympathetic nerves, n_{act}, nᵣₑₛₚᵢᵣ, nₛₜᵣₑₛₛ, n_{infl} and nₒₜₕₑᵣₛ respectively represent the neural afferent signals generated by movement, breathing, psychological stress, inflammation, and other internal or external factors, obtained through data acquired by the at least one miniature wearable vital sign acquisition device, C_{NE}, and C_{ACh} represent the gain of atrial sinus receptors, and HR_{Intrinsic} represents the intrinsic heart rate.

Therefore, according to this embodiment, the present invention innovatively proposes the establishment of a neurophysiological mathematical model for the regulation of heart rate by exercise, the regulation of heart rate by breathing, and the impact of stress and inflammation on heart rate. By calculating heart rate variability while considering various internal and external influencing factors, it reflects the regulatory state of sympathetic and parasympathetic nerves, thus providing practical reference significance for human health and disease.

In one embodiment, the specific test includes a digital walking test, and the multi-vital-sign data integrative analysis involves integrative analysis based on the physiological mathematical model of at least the motion and heart rate data of the user acquired by the at least one miniature wearable vital sign acquisition device during the digital walking test, to obtain the index of cardiac strain response (CR) for evaluating sympathetic nerve regulation ability and the index of cardiac strain inhibition (CI) for evaluating parasympathetic nerve inhibition ability. In a specific embodiment, the digital walking test lasts for a total of 7 minutes or 10 minutes, including guiding the user to rest for 2 minutes first, then walk as fast as possible for 3 minutes or 6 minutes, and rest for another 2 minutes after stopping walking. In a specific embodiment, the integrative analysis comprises: solving the following formula based on the sequences of motion intensity and heart rate: n_{act}(t), HR_{walking}(t), t={t_{walkstart-1}, t_{walkstop}}], which are acquired during the digital walking test, to obtain regulatory ability of sympathetic nerve on heart rate increase driven by motion, which is represented by AₛfₛC_{NE}, that is, the cardiac strain response CR:

$$H {R}_{walking} ( t ) - H {R}_{rest} = H {R}_{Intrinsic} {A}_{s} {f}_{s} ( {n}_{act} ( t ) - 1 ) {C}_{NE} \qquad ( 4 )$$

Wherein HR_{walking}(t) represents the heart rate that varies with time (t) during walking, HRᵣₑₛₜ represents the resting heart rate, and n_{act}(t) represents the exercise intensity that varies with time (t) during walking, expressed in metabolic equivalents; and solve the following formula based on the sequences [n_{act}(t), HR_{walking}(t), t={t_{walkstart-1}, t_{walkstop}}] of exercise intensity and heart rate acquired during the digital walking test to obtain the heart rate decline rate under the braking effect of the parasympathetic nerve after the cessation of exercise, represented by AₚfₚC_{ACh}, i.e., cardiac strain inhibition CI:

$${dHR}_{stop} ( t ) / dt = H {R}_{Intrinsic} {A}_{p} {C}_{ACh} {f}_{p} ( {n}_{act} ( {t}_{stop} ) - 1 ) \qquad ( 5 )$$

Wherein, HRₛₜₒₚ(t) represents the heart rate that changes with time (t) after the cessation of exercise, and tₛₜₒₚ represents the time after the cessation of exercise.

Therefore, according to the above embodiment, by guiding user through specific walking test, neural afferent is dominated by movement, and other factors can be neglected. In this way, from the physiological mathematical model of movement as neural afferent, excitation through the sympathetic nerve, and action on atrial sinus receptors to cause an increase in heart rate, the cardiac strain response CR and its optimization calculation formula are derived. In addition, this invention establishes a physiological mathematical model of parasympathetic nerve regulation of heart rate when movement ceases, and provides a calculation method for cardiac strain inhibition CI. Therefore, this not only improves the calculation method in physiological mathematical theory but also ensures the requirements for clinical application.

In one embodiment, the user APP comprises an activity evaluation unit configured to perform the digital walking test and provide personalized exercise training guidance and monitoring. The personalized exercise training program is generated and recommended by the data service based on the results of multi-vital-sign data integrative analysis based on the data from the digital walking test. In a specific embodiment, the personalized exercise training includes exercise training guided by a target heart rate, wherein, when the exercise heart rate does not reach the lower limit of the target heart rate, the user is reminded to increase the exercise intensity; when the exercise heart rate exceeds the upper limit of the target heart rate, the user is reminded to reduce the exercise intensity; and when the exercise duration is reached, the user is reminded to end the exercise training and a training report is generated. Wherein, the upper limit of the target heart rate is the maximum heart rate during the digital walking test, i.e., HRᵤₚ=HRₘₐₓ and the lower limit of the target heart rate is HR_{Low}=HRᵤₚ−CR×1 MET, wherein, CR×1 MET is the heart rate change value corresponding to an exercise intensity of 1 MET

### (Metabolic Equivalent Task).

Therefore, based on the aforementioned embodiments, personalized exercise training is guided by vital signs derived from specific walking tests, thereby providing customized training guidance and monitoring tailored to individual circumstances.

In one embodiment, the specific test comprises a breathing test, and the multi-vital-sign data integrative analysis comprises integrative analysis based on the physiological mathematical model of at least heart rate and PPG data of the user acquired by the at least one miniature wearable vital sign signal acquisition device during the breathing test, to obtain vagal tone VA and stress factor SF. In a specific embodiment, the breathing test lasts for 9 minutes and includes three stages: in the first stage, the user is guided to breathe freely in a resting state for 3 minutes; in the subsequent second stage, the user is guided to perform rhythmic breathing at a rate of 9 breaths per minute for 3 minutes; and in the subsequent third stage, the user is guided to perform rhythmic breathing at a rate of 6 breaths per minute for 3 minutes. In a specific embodiment, the integrative analysis based on the physiological mathematical model comprises: estimating respiratory rate based on the PPG data acquired during the breathing test; and solving the following autonomic cardiopulmonary regulation equation based on the respiratory rate and heart rate data to obtain vagal tone VA represented by AₚfₚC_{Ach}.

$$H R ( t ) = H {R}_{Intrinsic} ( 1 + {A}_{s} {f}_{s} ( {n}_{stress} , {n}_{infl} , {n}_{others} ) {C}_{NE} + {A}_{p} {f}_{p} ( {n}_{respir} ( t ) ) {C}_{ACh} ) \qquad ( 6 )$$

The step comprises applying band-pass filtering to the heart rate variations acquired during each stage of the breathing test based on the specified breathing frequency. Consequently, the above autonomic neurocardiovascular regulation equation (6) is rewritten as:

$${dHR}_{respir} ( t ) / dt = {A}_{p} {C}_{ACh} {df}_{p} ( {n}_{respir} ( t ) ) / dt \qquad ( 7 )$$

Wherein, the left side of formula (7) represents the heart rate changes caused by breathing during the breathing test, while the right side nᵣₑₛₚᵢᵣ(t) represents the respiratory signal. By optimally solving equation (7), the vagal tone VA=AₚfₚC_{ACh} can be obtained. Furthermore, after solving the heart rate variability caused by breathing in equation (6), the remaining AC spectrum in the frequency spectrum analysis of the heart rate variability sequence, after removing the heart rate variability component caused by breathing, is the stress factor SF related to psychological stress, inflammation, and other internal and external factors:

$$S F = \{ \sum [ {P}_{HR} ( f ) - {P}_{HR - respir} ( f ) ] \} / \sum {P}_{HR} ( f ) \qquad ( 8 )$$

Wherein, P_{HR}(f) is the Fourier transform domain power spectrum of the heart rate sequence, P_{HR-respir}(f) represents the Fourier transform domain power spectrum of heart rate changes resulting from the regulation of neural afferent through the vagus nerve during respiration. In a specific embodiment, the self-regulation score for each stage of the breathing test is calculated based on the values of vagal tone VA and stress factor SF for that stage using the following formula:

$$self - regulation score = [ \frac{VA}{nomal value} + ( 1 - S F ) ] / 2 \times 100 \qquad ( 9 )$$

And the overall self-regulation score is the average of the scores from the three stages of the breathing test.

Therefore, based on the above-mentioned embodiments, the present invention proposes a specific breathing test and two main indicators derived from this test: vagal tone (VA) and stress factors (SF). These indicators are accompanied by a dedicated scientific physiological mathematical model and calculation method for respiratory heart rate self-regulation, as well as clear clinical significance representing vagal tone and stress factors primarily consisting of psychological stress and inflammatory factors. Consequently, the method not only improves the calculation method in physiological mathematical theory but also ensures the requirements for clinical application.

In one embodiment, the user APP includes an autonomous regulation and evaluation unit, which is configured to perform the breathing test and provide personalized rhythmic deep breathing training guidance and monitoring. The personalized rhythmic deep breathing training program is generated and recommended by the data service based on the results of multi-vital-sign data integrative analysis based on the data from the breathing test.

Therefore, according to this embodiment, personalized rhythmic deep breathing training is provided based on the results of the breathing test, which is safe, effective, and beneficial to health.

In one embodiment, the miniature wearable vital sign acquisition device is a smart ring device, which includes: a light sensor unit comprising three light emitters for acquiring PPG signals and calculating heart rate and blood oxygen; a motion sensor unit comprising a three-dimensional acceleration sensor for acquiring and calculating user's motion data; a body temperature sensor unit for acquiring body temperature; a signal acquisition and transmission unit that acquires data from each sensor unit and transmits it to the use APP via Bluetooth; and a power management unit that provides stable voltage, monitors power level, and manages the charging process; wherein, all the above units are composed of separate dedicated chips.

Therefore, according to this embodiment, a separate dedicated chip is used to implement the smart ring, simplifying its design and ensuring stable operation.

In one embodiment, the user APP is capable of performing sleep staging based on the multi-vital-sign data integrative analysis and providing digital scores for sleep, activity, self-regulation, and health status. The data service side can interact with the user APP and provide data services including managing users and their data, organizing user information and databases, analyzing individual users' health status, scoring and development, conducting big data analysis based on group user data, and providing training prescription recommendation, management, and tracking services. In a specific embodiment, the user APP includes a health status scoring unit configured to: perform integrative analysis on heart rate and motion sequence data, remove data elements with a motion intensity greater than 1 expressed in metabolic equivalent, obtain a sequence of resting state heart rate data, and calculate the time domain parameters SDNN and RMSSD of heart rate variability (HRV) and the frequency domain normalized parameters LF and HF using a moving window; and calculate the health status score based on the average of scores in the following four aspects: activity score, sleep score, self-regulation score, and resting state heart rate variability (HRV) score. In a specific embodiment, the user APP includes a sleep staging and scoring unit configured to: perform integrative analysis on PPG, heart rate, heart rate variability, and motion data to classify sleep stages as awake, light sleep, deep sleep, and rapid eye movement; and score sleep in the following six dimensions, with a maximum score of 100:
1) Sleep duration accounts for 20 points. The reference sleep duration is 6-8 hours. If the sleep duration falls within the reference range, it is awarded 20 points; otherwise, it is awarded 10 points;
2) The deep sleep proportion accounts for 30 points. The reference proportion is that deep sleep accounts for 20% to 60% of the total sleep duration. Let the measured deep sleep proportion be x, then the deep sleep score=15+15*(x−20%)/40%;
3) The proportion of light sleep accounts for 20 points. The reference proportion is that light sleep accounts for less than 55% of the total sleep duration. Assuming the measured proportion of light sleep is x, then the light sleep score=18-10*(x−55%);
4) The number of arousals accounts for 10 points, with a reference range of 0 to 1 arousal. In the case of N arousals, the score=10-N*2;
5) The proportion of rapid eye movement accounts for 10 points. The reference proportion is 10-30%. If it falls within this range, 10 points will be given; otherwise, 5 points will be given
6) Sleep regularity is worth 10 points. The optimal bedtime is usually 22:00. Sleeping before 24:00 is worth 5 points. If the sleep time remains unchanged for more than 5 consecutive days within a half-hour range, an additional 5 points will be awarded.

In a preferred embodiment, the user data acquired by the at least one miniature wearable vital sign acquisition device further includes the user's body temperature, and the health status scoring unit implements body temperature physiological cycle calculation based on the body temperature.

Therefore, based on the aforementioned embodiments, the sleep staging utilizing multi-vital-sign data integrative analysis proposed by this invention provides digital scores for sleep, activity, self-regulation, and health status, as well as physiological cycle calculation, thereby offering users comprehensive personalized health monitoring.

The second aspect of the present invention provides a method for physical and mental health monitoring and services, comprising: acquiring and/or calculating at least the following data of a user through at least one miniature wearable vital sign acquisition device: heart rate, exercise, blood oxygen, and PPG; sending the acquired and/or calculated data to a user-end APP in the form of raw data and/or processed data through the at least one miniature wearable vital sign acquisition device; and analyzing the received data through the user APP; wherein the method comprises guiding the user to perform specific tests through the at least one miniature wearable vital sign acquisition device and/or the user APP, and performing multi-vital-sign data integrative analysis based on the data acquired and/or calculated during the specific tests through the user APP.

In one embodiment, the multi-vital-sign data integrative analysis is based on the established physiological mathematical model of autonomic nervous heart rate regulation that integrates multiple vital signs signals, expressed as the following formula:

$${M}_{s} = {A}_{s} {f}_{s} ( {n}_{act} , {n}_{respir} , {n}_{stress} , {n}_{infl} , {n}_{others} ) \qquad ( 10 )$$

$${M}_{p} = {A}_{p} {f}_{p} ( {n}_{act} , {n}_{respir} , {n}_{stress} , {n}_{infl} , {n}_{others} ) \qquad ( 11 )$$

$$H R = H {R}_{Intrinsic} ( 1 + {M}_{S} {C}_{NE} + {M}_{P} {C}_{ACh} ) \qquad ( 12 )$$

Wherein, Mₛ and Mₚ represents the neurotransmitters of sympathetic and parasympathetic nerves, n_{act}, nᵣₑₛₚᵢᵣ, nₛₜᵣₑₛₛ, n_{infl} and nₒₜₕₑᵣₛ respectively represent the neural afferent signals generated by movement, breathing, psychological stress, inflammation, and other internal or external factors, obtained through data acquired by the at least one miniature wearable vital sign acquisition device, C_{NE} and C_{ACh} represents the gain of atrial sinus receptors, and HR_{Intrinsic} represents the intrinsic heart rate.

In one embodiment, the specific test includes a digital walking test, and the multi-vital-sign data integrative analysis involves integrative analysis based on the physiological mathematical model of at least the movement and heart rate data of the user acquired by the at least one miniature wearable vital sign acquisition device during the digital walking test, to obtain the index of cardiac strain response (CR) for evaluating sympathetic nerve regulation ability and the index of cardiac strain inhibition (CI) for evaluating parasympathetic nerve inhibitory ability. In a specific embodiment, the digital walking test lasts for a total of 7 minutes or 10 minutes, including guiding the user to rest for 2 minutes first, then walk as fast as possible for 3 minutes or 6 minutes, and rest for another 2 minutes after the walking stops. In a specific embodiment, the integrative analysis based on the physiological mathematical model involves solving the following formula based on the sequences n_{act}(t), HR_{walking}(t), t={t_{walkstart-1}, t_{walkstop}}] of movement intensity and heart rate acquired during the digital walking test to obtain regulatory ability of the sympathetic nerve on heart rate elevation driven by movement represented by AₛfₛC_{NE}, that is, the cardiac strain response CR:

$$H {R}_{walking} ( t ) - H {R}_{rest} = H {R}_{Intrinsic} {A}_{s} {f}_{s} ( {n}_{act} ( t ) - 1 ) {C}_{NE} \qquad ( 13 )$$

Wherein, HR_{walking}(t) represents the heart rate variation with time (t) during walking, HRᵣₑₛₜ represents the resting heart rate, and n_{act}(t) represents the exercise intensity variation with time (t) during walking, expressed in metabolic equivalents; and solve the following formula based on the sequences [n_{act}(t), HR_{walking}(t), t={t_{walkstart-1}, t_{walkstop}}] of exercise intensity and heart rate collected during the digital walking test to obtain the heart rate decline rate presented by AₚfₚC_{ACh} under the braking effect of the parasympathetic nerve after the cessation of exercise, that is ardiac response inhibition CI:

$${dHR}_{stop} ( t ) / dt = H {R}_{Intrinsic} {A}_{p} {C}_{ACh} {f}_{p} ( {n}_{act} ( {t}_{stop} ) - 1 ) \qquad ( 14 )$$

Wherein, HRₛₜₒₚ(t) represents the heart rate that changes with time (t) after the cessation of exercise, and tₛₜₒₚ represents the time after the cessation of exercise.

In one embodiment, the method further comprises providing personalized exercise training guidance and monitoring based on the results of multi-vital-sign data integrative analysis conducted based on the data from the digital walking test. In a specific embodiment, the personalized exercise training includes exercise training guided by a target heart rate, wherein: when the exercise heart rate does not reach the lower limit of the target heart rate, the user is reminded to increase the exercise intensity; when the exercise heart rate exceeds the upper limit of the target heart rate, the user is reminded to reduce the exercise intensity; and when the exercise duration is reached, the user is reminded to end the exercise training and a report on the exercise training is generated; wherein the upper limit of the target heart rate is the maximum heart rate during the digital walking test, i.e., HRᵤₚ=HRₘₐₓ and the lower limit of the target heart rate is HR_{Low}=HRᵤₚ−CR×1 MET, where CR×1 MET represents the heart rate change value corresponding to an exercise intensity of 1 MET (Metabolic Equivalent Task).

In one embodiment, the specific test includes a breathing test, and the multi-vital-sign data integrative analysis involves integrative analysis based on the physiological mathematical model of at least heart rate and PPG data acquired by the at least one miniature wearable sign signal acquisition device during the breathing test, to obtain vagal tone VA and stress factor SF. In a specific embodiment, the breathing test lasts for a total of 9 minutes and includes three stages: in the first stage, the user is guided to breathe freely at rest for 3 minutes; in the subsequent second stage, the user is guided to perform rhythmic breathing at a rate of 9 breaths per minute for 3 minutes; and in the subsequent third stage, the user is guided to perform rhythmic breathing at a rate of 6 breaths per minute for 3 minutes. In a specific embodiment, the integrative analysis based on the physiological mathematical model involves estimating the breathing frequency based on the PPG data acquired during the breathing test; and solving the following autonomic cardiopulmonary regulation equation based on the breathing frequency and heart rate data to obtain vagal tone VA represented by AₚfₚC_{ACh}.

$$H R ( t ) = H {R}_{Intrinsic} ( 1 + {A}_{s} {f}_{s} ( {n}_{stress} , {n}_{infl} , {n}_{others} ) {C}_{NE} + {A}_{p} {f}_{p} ( {n}_{respir} ( t ) ) {C}_{ACh} ) \qquad ( 15 )$$

The step comprises applying band-pass filtering to the heart rate variations acquired during each stage of the breathing test based on the specified breathing frequency. Consequently, the above autonomic neurocardiovascular regulation equation (15) is rewritten as:

$${dHR}_{respir} ( t ) / dt = {A}_{p} {C}_{ACh} {df}_{p} ( {n}_{respir} ( t ) ) / dt \qquad ( 16 )$$

In the equation, the left side of formula (16) represents the heart rate changes caused by breathing during the breathing test, while the right side nᵣₑₛₚᵢᵣ(t) represents the breathing signal. By optimally solving equation (16), the vagal tone VA=AₚfₚC_{ACh} can be obtained. Furthermore, after solving the heart rate variability caused by breathing in equation (15), the remaining AC spectrum in the frequency spectrum analysis of the heart rate variability sequence, after removing the heart rate variability component caused by breathing, is the stress factor SF related to psychological stress, inflammation, and other internal and external factors:

$$S F = \{ \sum [ {P}_{HR} ( f ) - {P}_{HR - respir} ( f ) ] \} / \sum {P}_{HR} ( f ) \qquad ( 17 )$$

Wherein, P_{HR}(f) is the Fourier transform domain power spectrum of the heart rate sequence, and P_{HR-respir}(f) represents the Fourier transform domain power spectrum of heart rate changes resulting from the regulation of neural afferent through the vagus nerve during respiration. In a specific embodiment, the self-regulation score for each stage of the breathing test is calculated based on the values of vagal tone VA and stress factor SF for that stage using the following formula:

$$self - regulation score = [ \frac{VA}{nomal value} + ( 1 - S F ) ] / 2 \times 100 \qquad ( 18 )$$

And the overall self-regulation score is the average of the scores from the three stages of the breathing test.

In one embodiment, personalized rhythmic deep breathing training guidance and monitoring are provided based on the results of multi-vital-sign data integrative analysis conducted on the data from the breathing test.

In one embodiment, the method further comprises performing sleep staging based on the multi-vital-sign data integrative analysis, and providing digital scores for sleep, activity, self-regulation, and health status. In a specific embodiment, providing a score for health status includes: performing integrative analysis on heart rate and motion sequence data, removing data elements with a motion intensity greater than 1 expressed in metabolic equivalent, obtaining a resting state heart rate data sequence, and calculating the time domain parameters SDNN and RMSSD of heart rate variability (HRV) and the frequency domain normalized parameters LF and HF using a moving window; and calculating the health status score as the average of scores in the following four aspects: activity score, sleep score, self-regulation score, and resting state heart rate variability (HRV) score. In a specific embodiment, performing sleep staging includes performing integrative analysis on PPG, heart rate, heart rate variability, and motion data to classify sleep stages as awake, light sleep, deep sleep, and rapid eye movement; and providing a sleep score includes scoring sleep in the following six dimensions, with a maximum score of 100:
1) Sleep duration accounts for 20 points. The reference sleep duration is 6-8 hours. If the sleep duration falls within the reference range, it is worth 20 points; otherwise, it is worth 10 points;
2) The deep sleep proportion accounts for 30 points. The reference proportion is that deep sleep accounts for 20% to 60% of the total sleep duration. Let the measured deep sleep proportion be x, then the deep sleep score=15+15*(x−20%)/40%;
3) The proportion of light sleep accounts for 20 points. The reference proportion is that light sleep accounts for less than 55% of the total sleep duration. Assuming the measured proportion of light sleep is x, then the light sleep score=18-10*(x−55%);
4) The number of arousal episodes is scored as 10 points, with a reference range of 0 to 1 episode. In cases where there are N arousal episodes, the score is calculated as 10−N*2;
5) The proportion of rapid eye movement accounts for 10 points. The reference proportion is 10-30%. Within this range, 10 points are awarded; otherwise, 5 points are awarded
6) Sleep regularity is worth 10 points. The optimal bedtime is usually 22:00. Sleeping before 24:00 is worth 5 points. If the sleep time remains unchanged for more than 5 consecutive days within a half-hour range, an additional 5 points will be awarded.

In a preferred embodiment, the user data acquired by the at least one miniature wearable vital sign acquisition device further includes the user's body temperature, and the health status scoring unit implements body temperature physiological cycle calculation based on the body temperature.

Therefore, according to the present invention, a method for physical and mental health monitoring and services is provided, which is particularly capable of offering innovative integrative analysis technology for multi-vital-sign information, thereby providing users with professional monitoring and services for physical and mental health.

The third aspect of the present invention provides a machine-readable medium on which instructions are stored, said instructions enabling the implementation of the method as described above when executed by at least one processor.

It should be noted that one or more features described in different aspects of the present invention can be combined with one or more features described in other aspects to form embodiments of the present invention, and different embodiments with corresponding features can also achieve corresponding technical effects. Therefore, the embodiments and possible effects described above are not exhaustive.

### BRIEF DESCRIPTION OF DRAWINGS

To better understand the present invention, a detailed description will be provided with reference to the following drawings:

FIG. 1 is a block diagram illustrating a system for physical and mental health monitoring and services, including a smart ring device, according to one embodiment of the present invention;

FIG. 2 is a structural schematic diagram illustrating a smart ring device for physical and mental health monitoring and services, according to one embodiment of the present invention.

### DETAILED DESCRIPTION

The specific embodiments of the present invention will be described in detail below. It should be noted that the embodiments described here are merely for illustration and are not intended to limit the invention. In the following description, a large number of specific details are elaborated to provide a thorough understanding of the invention. However, it is apparent to those skilled in the art that the implementation of the technical solutions of the present invention does not necessarily require these specific details. In other embodiments, well-known structures, materials, or methods are not specifically described to avoid obscuring the invention.

Throughout the specification, references to “an embodiment”, “embodiment”, “an example”, or “example” imply that the specific features, structures, or characteristics described in conjunction with that embodiment or example are encompassed in at least one embodiment of the present invention. Therefore, the phrases “in one embodiment”, “in embodiments”, “an example”, or “example” appearing throughout the specification do not necessarily all refer to the same embodiment or example.

Furthermore, specific features, structures, or characteristics may be combined in any appropriate combination and/or sub-combination in one or more embodiments or examples. Additionally, those skilled in the art should understand that the term “and/or” as used herein encompasses any and all combinations of one or more of the listed items.

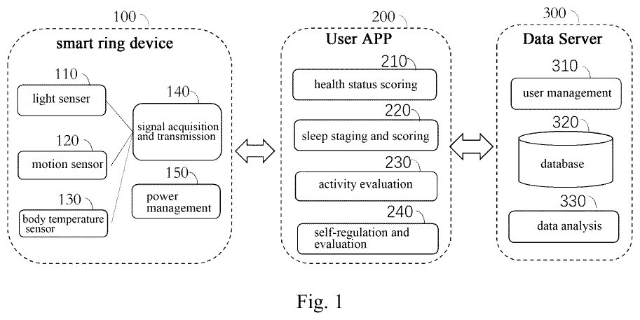

Next, referring to the block diagram shown in FIG. 1, we will describe a system for physical and mental health monitoring and services, including a smart ring, according to one embodiment of the present invention.

### Smart Ring Device 100

According to the present invention, a system for physical and mental health monitoring and services comprises a smart ring device 100 for acquiring vital signs.

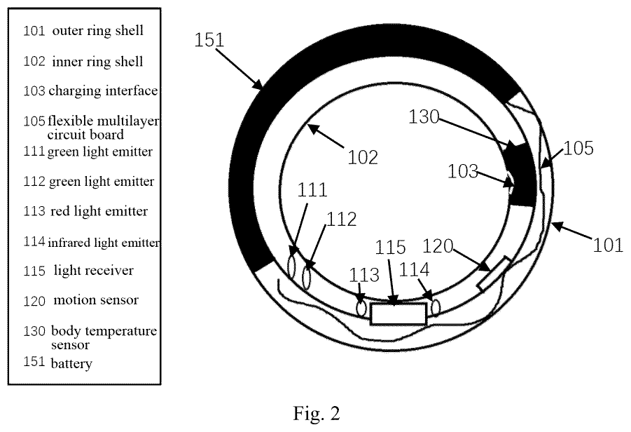

In one embodiment, the smart ring device 100 comprises a light sensor unit 110, a motion sensor unit 120, a body temperature sensor unit 130, a signal acquisition and transmission unit 140, and a power management unit 150. For the specific structure of the smart ring device 100, reference can also be made to the structural schematic diagram of the smart ring device 100 shown in FIG. 2.

According to the present invention, with the development of chip integration technology, these units can be implemented by commercially available dedicated chips that are available in existing technology.

According to the present invention, the light sensor unit 110 can be any dedicated chip for optical biosensing applications such as heart rate monitoring (HRM) and blood oxygen saturation (SpO₂) measurement, such as a medical analog front-end (AFE) device. In one embodiment, the light sensor unit 110 can include one or more light emitters and one or more light receivers to achieve biosensing measurements through the emission and reception of light signals. In one embodiment, the light emitters included in the light sensor unit 110 can emit any light that enables optical heart rate monitoring. For example, the light emitters included in the light sensor unit 110 can be 3-channel LED light emitters, namely light emitters 111 and 112 for emitting green light at 505-520 nm, light emitter 113 for emitting red light at 660 nm, and light emitter 114 for emitting infrared light at 940 nm. Similarly, in one embodiment, the light receivers included in the light sensor unit 110 are light receivers that enable the reception of any light emitted by the light emitters and reflected or transmitted by the human body (skin). In one embodiment, the light receivers included in the light sensor unit 110 can be a single light receiver 115 with a spectral range of 400-1100 nm or multiple light receivers each with a different spectral range. In one embodiment, the light sensor unit 110 can be integrated with embedded signal acquisition and processing algorithms to process the acquired signals to obtain heart rate and blood oxygen values. In one embodiment, the AFE4404 chip from Texas Instruments® can be used as the light sensor unit 110. In another embodiment, the GH3011 chip from Goodix can be used as the light sensor unit 110.

According to the present invention, the motion sensor unit 120 can be any dedicated chip for acquiring motion states, such as a three-axis MEMS (Micro-Electro-Mechanical Systems) accelerometer. In one embodiment, the LIS2DS12TR chip from STMicroelectronics® can be used as the motion sensor unit 120.

According to the present invention, the body temperature sensor unit 130 can be any dedicated chip for acquiring and outputting body temperature, such as a digital temperature sensor. In one embodiment, the TMP117MAIDRVR chip from Texas Instruments® can be used as the body temperature sensor unit 130.

According to the present invention, the signal acquisition and transmission unit 140 can be any dedicated chip that enables the acquisition/sampling of data from the light sensor unit 110, motion sensor unit 120, and body temperature sensor unit 130, as well as the transmission and reception of such data. In one embodiment, the signal acquisition and transmission unit 140 can sample data such as heart rate, blood oxygen, motion, and body temperature from the light sensor unit 110, motion sensor unit 120, and body temperature sensor unit 130, timestamp these data, and cache them locally and/or transmit them to the user APP 200. In one embodiment, the signal acquisition and transmission unit 140 can be integrated with a microprocessor to process signals from the light sensor unit 110, motion sensor unit 120, and body temperature sensor unit 130. In one embodiment, the signal acquisition and transmission unit 140 can utilize Bluetooth® protocols for data transmission, such as BLE protocol, Bluetooth 5.1 protocol, etc. In one embodiment, when the user APP 200 is connected to the smart ring device 100 via Bluetooth, the signal acquisition and transmission unit 140 can enable synchronization of time, uploading of data, receiving of instructions, etc. between the two. In one embodiment, the AMA3B2KK-KBR chip from Ambiq can be used as the signal acquisition and transmission unit 140. In another embodiment, the GR5515GGBD chip from Goodix can be used as the signal acquisition and transmission unit 140.

According to the present invention, the power management unit 150 can be any dedicated chip that enables power management, as is known in the art. In one embodiment, chips such as ON Semiconductor®'s regulated power supply chips NCP170AMX330TCG and NCP170AMX180TCG, Analog Devices' battery charging management chip LTC4065, and Texas Instruments®'s charging protection chip BQ29702DSER can be used for the power management unit 150. In another embodiment, chips such as Sage Micro®'s battery charging management chip SGM4056 and iCM-SEMI's charging protection chip CM1124 can also be used.

Of course, as those skilled in the art would easily appreciate, the dedicated chips used in the smart ring device 100 can also adopt other existing or future-developed chips with the same or similar functions, or any combination thereof.

FIG. 2 illustrates the structural schematic of a smart ring device 100 for physical and mental health monitoring and services, according to one embodiment of the present invention. As shown in FIG. 2, the smart ring device 100 comprises an outer ring shell 101, an inner ring shell 102, a charging interface 103, and a battery 151. In one embodiment, the charging interface 103 adopts a magnetic connection to ensure connection stability during charging. Furthermore, the smart ring device 100 also includes a flexible multilayer circuit board 105, on which various electronic circuits and chips included in the smart ring device 100 are integrated, such as a light sensor unit 110, a motion sensor unit 120, a body temperature sensor unit 130, a signal acquisition and transmission unit 140, and a power management unit 150, whose positions are shown in the figure, for example.

As mentioned earlier, the light sensor unit 110 can include one or more light emitters and one or more light receivers. For example, the light sensor unit 110 shown in FIG. 2 includes two green light emitters 111 and 112, a red light emitter 113, an infrared light emitter 114, and a light receiver 115. The inner layer of the finger ring where the light emitters and light receivers are located can be embedded with a transparent cover.

Of course, as those skilled in the art would readily appreciate, the specific structure of the smart ring device 100 is not limited to the specific structure described above and illustrated in FIG. 2. Changes can be made to the position, quantity, placement, and the like of the various components and devices included in the smart ring device 100 without departing from the scope of the present invention.

Furthermore, as those skilled in the art would readily appreciate, the devices used to acquire vital signs in systems for physical and mental health monitoring and services are not limited to smart ring devices, but can also be other forms of miniature wearable vital sign signal acquisition devices, such as smart wristband devices and smart watch devices.

### User APP 200

Referring back to FIG. 1, the system for physical and mental health monitoring and services according to one embodiment of the present invention further includes a user APP 200.

According to the present invention, the user APP 200 can receive PPG signals, heart rate, blood oxygen, motion, and body temperature data from the smart ring device 100 via Bluetooth, and utilize these data to fulfill various functions. For instance, the user APP 200 can connect to the smart ring device 100 (e.g., to its signal acquisition and transmission unit 140) via Bluetooth based on user requirements and issue data acquisition and transmission instructions. Furthermore, leveraging the aforementioned data it receives, the user APP 200 can process, integrate, and analyze multi-vital-sign data based on physiological mathematical models, compute various indicators and scores such as sleep, motion, and autonomic regulation, and ultimately derive a health status score.

On the other hand, the user APP 200 can also connect to the data server 300 via WiFi, cellular network, etc., upload data and analysis results to it, and receive further analysis and big data analysis results as well as service instructions from it.

In the analysis of vital sign data and health monitoring and services, traditional methods often analyze certain vital sign data in isolation. For instance, heart rate variability (HRV) is used to analyze heart rate data, but the neurophysiological regulation principles of heart rate changes are overlooked. Specifically, changes in heart rate are generated by various internal and external factors such as exercise, psychology, inflammation, and breathing, which are converted into neural afferent signals by sensory receptors and then outputted through the sympathetic and parasympathetic nervous systems as neurotransmitters, regulating the atria and sinus. This can be simplified and represented by the following formula:

$${M}_{s} = {A}_{s} {f}_{s} ( {n}_{act} , {n}_{respir} , {n}_{stress} , {n}_{infl} , {n}_{others} ) \qquad ( 1 )$$

$${M}_{p} = {A}_{p} {f}_{p} ( {n}_{act} , {n}_{respir} , {n}_{stress} , {n}_{infl} , {n}_{others} ) \qquad ( 2 )$$

$$H R = H {R}_{Intrinsic} ( 1 + {M}_{S} {C}_{NE} + {M}_{P} {C}_{ACh} ) \qquad ( 3 )$$

Wherein, Mₛ and Mₚ are neurotransmitters of the sympathetic and parasympathetic nerves, n_{act}, nᵣₑₛₚᵢᵣ, nₛₜᵣₑₛₛ, n_{infl}, nₒₜₕₑᵣₛ which are generated by neural afferent inputs from activity, breathing, psychological stress, inflammation, and other internal or external factors, respectively. C_{NE} and C_{ACh} represent the gain of atrial sinus receptors. HR_{Intrinsic} is intrinsic heart rate. That is to say, multiple neural afferent inputs stimulate the sympathetic and parasympathetic nerves, respectively producing neurotransmitters norepinephrine (NE) and acetylcholine (ACh), as shown in formulas (1) and (2). Changes in heart rate are generated by the action of neurotransmitters from the sympathetic and parasympathetic nerves Mₛ and Mₚ on the atrial sinus receptors, based on the intrinsic heart rate, as shown in formula (3).

Therefore, heart rate variability (HRV) is influenced not only by the regulatory state of sympathetic and parasympathetic nerves, but also by neural afferent signals generated by various internal and external factors. Thus, HRV calculated without considering these internal and external influencing factors cannot reflect the regulatory state of sympathetic and parasympathetic nerves, and therefore does not have much reference value for human health and disease.

Based on the above situation, this invention innovatively proposes the establishment of neurophysiological mathematical models for heart rate regulation by exercise, heart rate regulation by breathing, and the impact of stress and inflammation on heart rate. It achieves the following functions in the integration of multi-vital-sign data analysis:
1) Synchronously displaying changes in heart rate and exercise intensity indicates the relationship between heart rate and changes in exercise intensity. When calculating and analyzing heart rate variability, only the sequence consisting of heart rates at rest, represented by a metabolic equivalent of 1, is selected to calculate heart rate variability at rest, making HRV more stable, comparable, and applicable.
2) The Digital Walking Test (DWT) evaluates the sympathetic nerve regulation ability index, Cardiac Response (CR), and the parasympathetic nerve inhibition ability index, Cardiac Inhibition (CI), providing targeted indicators for the precise diagnosis and rehabilitation of patients with chronic diseases such as sympathetic overactivity hypertension, diabetes, and heart failure. Furthermore, a digital walking test guided by a smart ring and an APP has been designed. Users are guided to rest for 2 minutes, then walk as quickly as possible for 3 or 6 minutes (optional), and rest for 2 minutes after completion. Since the increase in heart rate from rest to walking is primarily driven by sympathetic nerve activation, a physiological mathematical model is solved based on the walking exercise intensity and heart rate data sequence to obtain the index for evaluating sympathetic nerve regulation ability: Cardiac Response (CR). When walking stops, the recovery of heart rate is braked by the parasympathetic nerve, and the corresponding physiological mathematical model is solved to obtain the index for evaluating parasympathetic nerve inhibition ability: Cardiac Inhibition (CI).
3) Guide and monitor exercise training with target heart rate. Through digital walking tests, personalized prescriptions for exercise training are recommended, with exercise intensity set based on the target heart rate. Guide and monitor exercise training with a smart ring and an APP. When the heart rate does not reach the lower limit of the target heart rate, remind the user to increase the exercise intensity; when the heart rate exceeds the upper limit of the target heart rate, remind the user to reduce the exercise intensity.
4) Breathing tests evaluate vagal tone (VA) and stress factors (SF), providing numerical measures for physical and mental health. Vagal tone is a targeted indicator for behavioral regulation and various chronic diseases. Guided breathing tests are conducted using a smart ring and an app: during resting state, guided breathing at 9 breaths per minute and 6 breaths per minute for 3 minutes each, totaling 9 minutes. The neural afferent of breathing regulates heart rate changes through the vagus nerve, medically known as respiratory sinus arrhythmia (RSA). As the rhythmic breathing frequency resonates with heart rate changes, the heart rate variation reaches its maximum. RSA occurs under the regulation of the vagus nerve, thus also measuring the tone of the vagus nerve. This invention establishes a physiological mathematical model of respiration regulating heart rate through the vagus nerve, and measures vagal tone (VA) and stress factors (SF) through breathing tests.
5) Guide and monitor personalized rhythmic deep breathing exercises. These exercises effectively lower blood pressure, improve mental health, and reduce erosion of chromosomal telomeres.
6) Multi-vital-sign data integrative analysis scientifically stages sleep, providing digital scores for sleep, activity, auto-regulation, and wellness.

Therefore, according to the present invention, the user-end APP 200 comprises a health status scoring unit 210, a sleep staging and scoring unit 220, an activity evaluation unit 230, and an autonomous regulation evaluation unit 240, providing users with physical and mental health monitoring, evaluation, and enhancement training.

### Wellness Scoring Unit 210

According to the present invention, the health status scoring unit 210 can perform vital sign monitoring and integrative analysis using physiological cycles as the time unit. The data acquired by the smart ring includes heart rate, movement, body temperature, blood oxygen, and respiratory signals separated from the PPG signal. According to the present invention, the original and innovative integrative of movement and heart rate analysis enables the analysis of heart rate variability in a resting state. Specifically, for heart rate and movement sequence data, remove movement state data, such as data elements with a movement intensity greater than 1 expressed in metabolic equivalents, to obtain a sequence of resting state heart rate data. A moving window is applied to this heart rate data sequence, and within the window, calculations of time domain parameters such as SDNN and RMSSD of heart rate variability (HRV), as well as frequency domain normalized parameters LF and HF, are completed.

In one embodiment, the wellness score is calculated as the average of scores from the following four aspects:
1) Activity: Percentage of daily exercise goals achieved;
2) Sleep score;
3) Auto-regulation score;
4) Heart rate variability (HRV) score under all-day resting state. Using the normal values of average heart rate, SDNN of heart rate variability, normalized LF, and HF as references, full marks are given within the normal range, and the further the deviation, the lower the score. Taking the average heart rate as an example, the normal range is 60-80 BPM. An average heart rate within this range scores 100 points, scores between 60-100 points within the range of 80-100 BPM, and scores below 60 points if it is higher than 100 BPM.

### Calculation of Body Temperature Physiological Cycle

According to the present invention, the health status scoring unit 210 can also calculate the physiological temperature cycle for a specific population, namely, women of appropriate age. During the menstrual period and a period of time after menstruation in adult women, the body maintains a low temperature level, approximately between 36.2° C. and 36.5° C. The temperature reaches its lowest point the day before ovulation, and after ovulation, the ovary forms a corpus luteum and begins to secrete progesterone, which causes the temperature to rise by about 0.5° C., reaching between 36.7° C. and 37.0° C., and remains in this temperature range for about 14 days until the next menstrual period.

Therefore, according to the present invention, the health condition scoring unit 210 can measure the user's basal body temperature data and plot a body temperature change curve. The menstrual period is determined and input by the user. The health condition scoring unit 210 can observe the body temperature changes from the day of cessation of menstruation, find the lowest point of body temperature, which is the ovulation day, and determine the menstrual period, safe period, ovulation period, and the current period. In one embodiment, the health condition scoring unit 210 can also mark the three periods and the current position with different colors.

### Sleep Staging and Scoring Unit 220

According to the present invention, the sleep staging and scoring unit 220 can integrate data such as PPG, heart rate, heart rate variability, and activity to perform sleep staging, including awake, light sleep, deep sleep, and rapid eye movement (N1 asleep, N2 light sleep, N3 deep sleep, REM rapid eye movement).

In one embodiment, the sleep score has a maximum of 100 and is evaluated based on six dimensions:
1) Sleep duration accounts for 20 points. The reference sleep duration is 6-8 hours. If the sleep duration falls within the reference range, it is worth 20 points; otherwise, it is worth 10 points.
2) The proportion of deep sleep accounts for 30 points. The reference proportion is that deep sleep accounts for 20% to 60% of the total sleep duration. Assuming the measured proportion of deep sleep is x, then the deep sleep score=15+15*(x−20%)/40%.
3) The proportion of light sleep accounts for 20 points, with a reference proportion of light sleep being less than 55% of the total sleep duration. Assuming the measured proportion of light sleep is x, the light sleep score=18-10*(x−55%).
4) The frequency of arousal counts for 10 points. The reference frequency is 0-1 time. In the case of arousal N times, the score=10-N*2.
5) The proportion of rapid eye movement accounts for 10 points. The reference proportion is 10-30%. If it falls within this range, score 10 points; otherwise, score 5 points.
6) Sleep regularity accounts for 10 points. The optimal bedtime is usually 22:00. Sleeping before 24:00 earns 5 points, and if the sleep time does not vary by more than half an hour for more than 5 consecutive days, an additional 5 points are awarded.

### Activity Evaluation Unit 230

According to the present invention, the activity evaluation unit 230 can measure the amount of exercise primarily based on steps. This can be calculated, for example, using the formula provided by the American Council on Exercise Medicine (ACSM):

The number of steps per minute is obtained from the signal acquisition and transmission unit 140 included in the smart ring device 100, and the user's step length is calculated by multiplying the user's height by 0.43, thereby calculating the movement speed S. Subsequently, the movement intensity and calorie consumption expressed in metabolic equivalent of task (METs) are calculated:

$$Walking : MET = 1 + ( 0.1 \times S ) / 3.5 + ( 1.8 \times S \times G ) / 3.5 \qquad ( 4 )$$

$$Running : MET = 1 + ( 0.2 ⨯ S ) / 3.5 + ( 0.9 ⨯ S ⨯ G ) / 3.5 \qquad ( 5 )$$

$$Calorie expenditure : KCAL / MIN = ( METs \times 3.5 \times M ) / 200 \qquad ( 6 )$$

In the above formula, S represents speed, with the unit of meter per minute; G denotes the gradient expressed as a percentage; and M signifies the body mass in kilograms.

Based on user's situation, set daily calorie consumption for activities. Score based on whether the activity goal is achieved on the same day. If the goal is achieved, score 100; if there is no activity within one hour, deduct 10 points.

The activity scoring only evaluates the user's activity status. In order to assess their motor function and the autonomic nervous system's ability to regulate and support motor function, the system according to the present invention provides a “digital walking test” function.

### Digital Walking Test (DWT), 2-3 (6)-2 Test Mode

The smart ring and accompanying app guide users to rest for 2 minutes first, then walk as quickly as possible for 3 or 6 minutes (adjustable), followed by another 2 minutes of rest after the walk.

In this digital walking test, the heart rate during the resting state for the first 2 minutes before exercise, the MET value of exercise intensity during 3 or 6 minutes of walking, and the corresponding heart rate, as well as the change in heart rate after the MET value decreases to the resting state value upon stopping walking, are calculated from the data provided by the signal acquisition and transmission unit 140 included in the smart ring device 100. During the entire digital walking test, when transitioning from rest to walking, the sudden increase in exercise intensity as a neural input rapidly increases sympathetic nerve excitation, leading to an increase in the concentration of its output transmitter norepinephrine, which stimulates atrial sinus receptors and increases heart rate. Upon stopping walking, the recovery of heart rate is primarily due to the braking effect of the parasympathetic nerve.

In the walking test, due to the dominance of motor nerve afferent, other factors can be neglected. From formulas (1) to (3), we can obtain the mathematical description of the neurophysiological changes in heart rate caused by the jump changes in walking start and stop intensities, regulated by sympathetic and parasympathetic nerves:

$$H {R}_{walking} ( t ) - H {R}_{rest} = H {R}_{Intrinsic} {A}_{s} {f}_{s} ( {n}_{a c t} ( t ) - 1 ) {C}_{N E} \qquad ( 7 )$$

$${dHR}_{s t o p} ( t ) / dt = {HR}_{Intrinsic} {A}_{p} {C}_{A C h} {f}_{p} ( {n}_{a c t} ( {t}_{s t o p} ) - 1 ) \qquad ( 8 )$$

Wherein, HR_{walking}(t) represents the heart rate changing with time (t) during walking, HR_{walking}(t) represents the resting heart rate, HRᵣₑₛₜ represents the intrinsic heart rate, HR_{Intrinsic} represents the exercise intensity changing with time (t) during walking, expressed in metabolic equivalents, HRₛₜₒₚ(t) represents the heart rate changing with time (t) after the cessation of exercise, and tₛₜₒₚ represents the time after the cessation of exercise.

Based on formula (7), from the sequences represented by n_{act}(t), HR_{walking}(t), t={t_{walkstart-1}, t_{walkstop}}] of exercise intensity metabolic equivalent values and exercise heart rate values, a quadratic sequence can be listed, and by using optimization methods, the regulatory ability of the sympathetic nervous system on heart rate elevation driven by exercise, can be solved, and an indicator name is assigned: Chronotropic Response (CR).

Similarly, according to formula (8), after the cessation of exercise, the heart rate decreases due to the braking effect, which is represented by AₚfₚC_{ACh}, of the parasympathetic nervous system. Its value is characterized by the rate of decrease. The indicator is named “Chronotropic Inhibit” (CI).

Therefore, the indicator series for the digital walking test is obtained:

| indicator name | clinical significance |
|---|---|
| Resting heart rate (HR) | Average resting heart rate in the |
| | first 2 minutes |
| Maximum heart rate during walking | Average maximum heart rate |
| test, HRmax | during walking |
| Maximum Metabolic Equivalent Task | Maximum exercise intensity |
| (METs) during walking test | during walking test |
| Resting blood pressure | |
| Walking distance | A numerical measure of athletic |
| | ability |
| Chronotropic Response CR | Sympathetic motor heart rate |
| | regulation |
| Chronotropic Inhibit (CI) | Parasympathetic heart rate |
| | inhibitory capacity |

The indicator series and the generated test report, including the recommended exercise training prescription, will be uploaded to the data server 300.

The Chinese patent titled “Device, System, and Method for Testing Cardiac Motion Function”, with US201610256974.5 and under the name of the inventor, records a walking test and defines the chronotropic rate (CR) as the change in heart rate caused by a unit change in motion equivalent, which is calculated by the following formula:

$$C R = ( H {R}_{S t a g e} - H {R}_{rest} ) / ( ME {T}_{s t a g e} - 1 ) \qquad ( 9 )$$

However, due to the noise present in the heart rate and motion data during the actual experiment, the heart strain rate calculated using the aforementioned formula exhibits instability.

Therefore, according to the present invention, a physiological mathematical model is derived from the perspective of exercise acting as neural input, stimulating the sympathetic nervous system, and affecting the atrial sinus receptors to cause an increase in heart rate. This model leads to the Chronotropic Response (CR) of cardiac strain and its optimized calculation formula (7). This undoubtedly represents an innovation in physiological mathematical theory and computational methods, while also ensuring the requirements for clinical application.

In addition, the patent “Device, System, and Method for Testing Cardiac Motion Function” also defines 1-minute heart rate recovery. However, due to noise present in heart rate and motion data during actual experiments, there is instability in the indicators. Therefore, this invention also proposes a new indicator based on the physiological mathematical model of the parasympathetic nervous system's inhibitory effect on heart rate during cessation of movement: Chronotropic Inhibit CI and its optimized calculation formula (8).

One-minute heart rate recovery is an intuitive definition (ad hoc) derived from clinical practice, lacking a physiological theoretical foundation. However, the present invention establishes a physiological mathematical model of parasympathetic regulation of heart rate at the cessation of exercise, and provides a calculation method for cardiac strain inhibition (CI), making the clinical significance of the indicator name clear. This undoubtedly represents a significant and substantial improvement over existing technology.

The digital walking test according to the present invention is simple and feasible, and can be conducted under the voice guidance of an APP. The test indicators according to the present invention have a solid physiological basis in neural regulation, and the measurement and calculation intelligence of the indicators have clear clinical significance: cardiac strain response (CR) and cardiac strain inhibition (CI) correspond to sympathetic and parasympathetic neural regulatory capabilities, respectively, and serve as digital evidence for diagnosing sympathetic overactivity-type hypertension, precise treatment of heart failure, diabetes, and other chronic diseases.

Guide and Monitor Exercise Training with Target Heart Rate

The system according to the present invention also provides personalized exercise training guidance and monitoring functions. Personalized exercise training is provided based on the vital sign data measured during the digital walking test:

Download personalized exercise training prescriptions recommended based on digital walking test results from the data server 300.

Guide and monitor exercise training with a smart ring and an APP. When the exercise heart rate does not reach the lower limit of the target heart rate, remind the user to increase the exercise intensity; when the exercise heart rate exceeds the upper limit of the target heart rate, remind the user to reduce the exercise intensity.

When the exercise duration is reached, remind the user to end the exercise training and generate an exercise training report.

According to the present invention, the upper limit of target heart rate is the maximum heart rate during the digital walking test, that is HRᵤₚ=HRₘₐₓ, and the lower limit of target heart rate is HR_{Low}=HRᵤₚ-CR×1 MET, wherein, CR×1 MET represents the heart rate change value corresponding to an exercise intensity of 1 MET.

### Self-Regulation and Evaluation Unit 240

### Breathing Test

According to the present invention, users can be guided to perform a breathing test in a resting state using a smart ring and an APP. In one embodiment, the breathing test involves free breathing for 3 minutes, followed by guided rhythmic breathing at 9 breaths per minute for 3 minutes, and then guided rhythmic breathing at 6 breaths per minute for 3 minutes, for a total of 9 minutes.

Breathing is the only way for the human body to influence autonomic nervous regulation. As a neural input, breathing regulates heart rate through the vagus nerve: inhalation increases heart rate, while exhalation slows it down. This phenomenon is known as “Respiration Sinus Arrhythmia (RSA)”. Therefore, the amplitude of RSA indicates the tension of the vagus nerve; according to the multi-layer vagal nerve theory, RSA is a representation of behavioral control ability. As the rhythmic breathing frequency decreases, this resonance phenomenon between breathing and heart rate intensifies, reaching its maximum when close to 6 breaths per minute. This phenomenon is called “Cardio Pulmonary Resonance (CPR)”.

However, chronic diseases such as heart failure and diabetes, psychological stress, and inflammation can be broadly referred to as “Stress Factor SF”, which are all interfering factors of cardiopulmonary resonance, affecting the amplitude of RSA.

Due to the lack of strong rhythmicity and insufficient depth of breathing in a state of free breathing, the resulting heart rate changes are not obvious. In order to better evaluate a person's “autonomous regulation” state, we introduce two additional stages of rhythmic deep breathing guided by the user's APP, at 9 breaths per minute and 6 breaths per minute, to form a complete breathing test process.

In the context of breathing testing, the equations (1) to (3) for autonomic neurocardiovascular regulation can be rewritten as follows:

$$HR ( t ) = H {R}_{Intrinsic} ( 1 + {A}_{s} {f}_{s} ( {n}_{stress} , {n}_{inf l} , {n}_{others} ) {C}_{N E} + {A}_{p} {f}_{p} ( {n}_{respir} ( t ) ) {C}_{A C h} ) \qquad ( 10 )$$

Wherein, breathing regulates heart rate through the vagus nerve (parasympathetic nerve), while psychological stress and inflammation affect heart rate through the sympathetic nerve. Since the vagus nerve is myelinated, transmission delay is minimal, allowing heart rate changes to follow the breathing rhythm, forming resonance. Stress factors such as psychological stress and inflammation are transmitted through sensory receptors and delayed by the sympathetic nerve, acting on atrial sinus receptors and blocking cardiopulmonary resonance. The long-term effects of heart failure and diabetes lead to pathological remodeling of the heart, lungs, and nervous system, greatly reducing the resonance characteristics of the cardiopulmonary and data systems.

Using the PPG signal obtained from the signal acquisition and transmission unit 140 included in the smart ring device 100, the respiratory signal is estimated. Together with the heart rate data, the vagal activity VA and stress factor SF are solved separately according to the cardiopulmonary regulation equation (10).

Since both the respiratory signal and the corresponding heart rate variation in formula (10) are rhythmic signals with similar frequencies, band-pass filtering can be applied to the heart rate variation based on the respiratory frequency during each stage of the breathing test. Formula (10) can be rewritten as:

$$d H {R}_{respir} ( t ) / dt = {A}_{p} {C}_{A C h} d {f}_{p} ( {n}_{respir} ( t ) ) / dt \qquad ( 11 )$$

The left side of the equation represents the heart rate changes caused by breathing during the breathing test, while the right side nᵣₑₛₚᵢᵣ(t) represents the respiratory signal.

Formula (11) indicates that the heart rate variability induced by breathing is consistent with the changing rhythm of respiratory nerve afferent; its amplitude can be obtained by optimizing the solution of formula (11) using respiratory and heart rate change sequence data: vagal tone VA=AₚfₚC_{ACh}.

After obtaining the heart rate variability caused by breathing in formula (10), in the spectral analysis of the heart rate variability sequence, the component of heart rate variability generated by breathing is removed, and the remaining alternating current spectrum represents the proportion of stress factors SF such as psychological pressure and inflammation. According to the present invention, the stress factor SF is:

$$S F = \{ \sum [ {P}_{H R} ( f ) - {P}_{HR - respir} ( f ) ] \} / \sum {P}_{H R} ( f ) \qquad ( 12 )$$

Wherein P_{HR}(f) is the Fourier transform domain power spectrum of the heart rate sequence, and P_{HR-respir}(f) represents the Fourier transform domain power spectrum of heart rate changes resulting from the regulation of neural afferent via the vagus nerve during respiration.

So far, based on the physiological mathematical model of vagal regulation represented by formulas (10) and (11), which describes the changes in respiratory rate and heart rate, the two key indicators in the breathing test, namely vagal tone VA and stress factor SF, have been solved using respiratory and heart rate sequences. The indicators generated during the three test phases of the breathing test are as follows: 1) average heart rate HR; 2) respiratory rate RR; 3) respiratory stability RS; 4) vagal tone VA; 5) stress factor SF.

The self-regulation score is calculated as the average of the scores for vagal tone (VA) and stress factor (SF) among the three stages of the breathing test. Specifically, the vagal tone score is calculated as 100 times the ratio of VA to the normal value, while the stress factor score is calculated as (1−SF) times 100. More precisely, the self-regulation score for each stage of the breathing test is determined based on the values of vagal tone (VA) and stress factor (SF) for that stage using the following formula:

$$self - regulation score = [ \frac{V A}{nomal value} + ( 1 - S F ) ] / 2 \times 100 \qquad ( 13 )$$

And the overall self-regulation score is the average of the scores from the three stages of the breathing test.

The series of cardiopulmonary system indicators disclosed in the Chinese patent application titled “Cardiopulmonary Respiration Test and Personalized Deep Breathing and Oxygen Therapy System and Equipment” (application Ser. No. 20/2111030099.6) which is under the name of the inventor, include: respiratory system indicator RSI, which specifically includes: main respiratory rate MR, respiratory smoothness RRR, tidal volume TV, and oxygen saturation OS; cardiovascular system indicator CSI, which specifically includes: mean heart rate MHR, heart rate variability standard deviation HRSD, heart rate power spectrum very low frequency component VLF, as well as mean blood pressure MBP, blood pressure variability standard deviation BPSD, and ambulatory arterial stiffness index AASI; cardiopulmonary interaction indicator CPII, which specifically includes: amplitude of respiratory heart rate variation AHR, respiratory heart rate modulation RCM, and respiratory heart rate correlation coefficient CRH, defined as follows:

$$AHR = {mean}_{K} \{ {max}_{RespCycle K} HR - {min}_{RespCycle} HR )$$

$$RCM = {\int }_{M R R - c}^{M R R + c} {F}_{Res p} ( n ) {F}_{H R} ( n ) / {\int }_{0}^{∞} {F}_{H R} ( n )$$

$$CRH = {max}_{m = 0 , M} [ C o r ( {f}_{r e s p} ( t + m ) , {f}_{H R} ( t ) ) / \sqrt{Var ( {f}_{r e s p} ( t + m ) ) Var ( {f}_{H R} ( t ) )} ]$$

However, unlike the indicators defined in the existing technology, the two main indicators of the breathing test proposed in this invention, vagal tone (VA) and stress factors (SF), are backed by a dedicated scientific physiological mathematical model and calculation method for respiratory heart rate autonomic regulation. At the same time, they possess clear clinical significance in representing vagal tone and stress factors primarily consisting of psychological stress and inflammatory factors. This undoubtedly represents a significant and substantial improvement over the existing technology.

According to the present invention, in addition to providing indicators for three test stages, the breathing test also recommends a personalized rhythmic deep breathing prescription, generates a test report, and uploads it to the data server 300.

The breathing test proposed in this invention utilizes a miniature wearable device, namely the smart ring device 100, combined with a user-end APP, making it convenient to use, fully intelligently guided, and with clear digital indicators. Numerous clinical studies have shown that the key indicators in this invention, namely vagal tone VA and stress factor SF, are targeted indicators for psychological disorders, cardiopulmonary diseases, and diabetes.

### Personalized Rhythmic Deep Breathing Training Guidance and Monitoring

The personalized rhythmic deep breathing training according to the present invention is safe, effective, and beneficial to health. For example:
Nobel Prize laureate Ignarro: Deep breathing generates nitric oxide (NO), which dilates blood vessels, prevents thrombosis, and effectively blocks the growth of bacteria and viruses.
Nobel Prize laureate Blackburn has found that deep breathing can reduce ineffective alveolar space, enhance the efficiency of pulmonary gas exchange, improve mitochondrial oxygen supply to brain cells, prolong cell lifespan and chromosome telomere length, and enhance gene epigenetics.
Research has shown that near a rhythmic breathing rate of 6 breaths per minute, “cardiopulmonary resonance” occurs, leading to an effective increase in vagal tone, resulting in a pleasant mood and agile thinking.

According to the present invention, the personalized rhythmic deep breathing training prescription TDFIT generated and recommended by the breathing test consists of the following five elements: breathing training frequency (Times per day), default value: once a day; training duration (Duration), default value: 10 minutes; breathing frequency (Frequency), based on the scores of three test phases, the highest recommended breathing frequency; inhalation-exhalation ratio (Inhalation-exhalation ratio), default value: 1.5; breathing type (Type): default type: pursed lip breathing, inhaling through the nose and exhaling through the mouth.

The personalized rhythmic deep breathing training provided by the system according to the present invention offers the following guidance and monitoring:
Download personalized rhythmic deep breathing training prescriptions generated and recommended by the breathing test from the data server 300.
According to the prescription, personalized rhythmic deep breathing exercises are guided and monitored by a smart ring and an APP.
Upon reaching the training duration, provide a reminder, terminate the training session, and generate a training report.

### III. Data Server 300

Continuing to refer to FIG. 1, the system for physical and mental health monitoring and services according to one embodiment of the present invention further includes a data service terminal 300 for providing data services. According to the present invention, the data service terminal 300 is capable of interacting with the user terminal APP 200, managing users, receiving data, organizing and managing user information and databases, performing big data analysis on multi-user data, analyzing the pre- and post-state of each user, as well as managing and tracking services, among other functions.

According to the present invention, the data server 300 comprises a user management unit 310, a database unit 320, and a data analysis unit 330.

In one embodiment, the functions of the user management unit 310 include user registration, member management, rights setting, service management, etc.

In one embodiment, the database unit 320 organizes user basic data, vital sign monitoring data, analysis results, and various scores with the user as the core, derives walking test and breathing test reports, and provides various feedback, exercise training, and execution reports of rhythmic deep breathing training.

In one embodiment, the data analysis module 330 primarily performs two major aspects of data analysis tasks:
1. For individual users, based on the basic data in the database, as well as monitoring data and various scores over a period of time, analyze the overall state and changing trends of the user's physical and physiological health, and provide the analysis to the user or share it with family members according to the set services. In addition, based on the reports of exercise training and rhythmic deep breathing training over a period of time, analyze their progress and provide prescription optimization suggestions.
2. For all users, based on their basic data, monitoring data, scores, walking test and breathing test reports, training progress, etc. in the database, user classification is conducted. Typical cases are selected for each category of users, and the health management situation of this type of users is summarized. Based on this, the optimal management plan is formulated. This provides theoretical and technical support for promoting and optimizing health management.

In summary, according to the present invention, a system for physical and mental health monitoring and services is proposed. The system may include a smart ring device 100 for acquiring vital signs, a user APP 200, and a data server 300 for providing data services. According to the present invention, the smart ring device 100 is used to acquire human vital signs signals such as PPG, heart rate, activity, body temperature, and blood oxygen, and transmit the acquired data to the user APP 200 through communication methods such as Bluetooth. Furthermore, according to the present invention, the user APP 200 performs various analyses and evaluations on the acquired data to provide users with physical and mental health monitoring, evaluation, and improvement services. In addition, the user APP 200 can also communicate with the data server 300 through communication methods such as WiFi, and upload data to the data server 300, so that the latter can provide services such as data management and data storage on the one hand, and further analyze the uploaded data and provide services such as analysis, scoring, and recommendation on the other hand.

According to the present invention, the analysis of human vital signs acquired by the smart ring device 100 specifically includes integrative analysis, which integrates human data such as PPG, heart rate, activity, body temperature, and blood oxygen to derive indicators and parameters with vital signs and clinical application value. Based on these parameters, professional applications for physical and mental health monitoring and services are realized.

The described aspects and examples of the present invention are intended to be illustrative and not restrictive, and are not intended to represent every aspect or example of the invention. Although the basic novel features of the invention as applied to various specific aspects of the invention have been shown, described, and pointed out, it will also be understood that those skilled in the art may make various omissions, substitutions, and changes in the form and details of the illustrated devices and their operation without departing from the spirit of the invention. For example, all combinations of those elements and/or method steps that are explicitly intended to perform essentially the same function in essentially the same way to achieve the same result are within the scope of the invention. Furthermore, it should be recognized that structures and/or elements and/or method steps shown and/or described in connection with any form or aspect of the invention may be incorporated into any other invention or described or suggested form or aspect as a general matter of design choice. Additionally, various modifications and variations can be made without departing from the spirit or scope of the invention as set forth in the appended claims and legally recognized equivalents thereof.

1. A system for physical and mental health monitoring and services, comprising:
at least one miniature wearable vital sign acquisition device, configured to acquire at least the following data of the user: heart rate, movement, blood oxygen and PPG, and send the acquired data to the a user APP in the form of raw data or processed data;
the user APP (200), which is configured to analyze the received data; and
a data server (300), which is configured to communicate with the user APP to provide data services;
wherein the system is configured to guide the user to conduct specific tests through at least one miniature wearable vital sign acquisition device or the user APP, and perform multi-vital-sign data integrative analysis based on the data acquired during the specific tests through the user APP.

2. The system according to claim 1, wherein the multi-vital-sign data integrative analysis is based on a physiological mathematical model of autonomic heart rate regulation that integrates multi-vital-sign signals, wherein the model is represented by the following formula:
$${M}_{s} = {A}_{s} {f}_{s} ( {n}_{act} , {n}_{respir} , {n}_{stress} , {n}_{infl} , {n}_{others} ) \qquad ( 1 )$$
$${M}_{p} = {A}_{p} {f}_{p} ( {n}_{act} , {n}_{respir} , {n}_{stress} , {n}_{infl} , {n}_{others} ) \qquad ( 2 )$$
$$HR = H {R}_{Intrinsic} ( 1 + {M}_{s} {C}_{N E} + {M}_{P} {C}_{ACh} ) \qquad ( 3 )$$
wherein Mₛ and Mₚ represent the neurotransmitters of the sympathetic and parasympathetic nerves, respectively, n_{act}, nᵣₑₛₚᵢᵣ, nₛₜᵣₑₛₛ, n_{infl} and nₒₜₕₑᵣₛ represent the neural afferent signals generated by movement, breathing, psychological stress, inflammation and other internal or external factors, respectively, obtained through data acquired by the at least one miniature wearable vital sign acquisition device, C_{NE} and C_{ACh} represent the gain of atrial sinus receptors, and HR_{Intrinsic} represents the intrinsic heart rate.

3. The system according to claim 2, wherein the specific test comprises a digital walking test, which takes 7 minutes or 10 minutes, wherein the digital walking test includes guiding the user to rest for 2 minutes, then walk as fast as possible for 3 minutes or 6 minutes, and rest for another 2 minutes after the walking stops; and the multi-vital-sign data integrative analysis includes integrative analysis of at least the motion and heart rate data of the user acquired by the at least one miniature wearable vital sign acquisition device during the digital walking test based on the physiological mathematical model, to obtain the index of cardiac strain response (CR) for evaluating sympathetic nervous system regulation ability and the index of cardiac strain inhibition (CI) for evaluating parasympathetic nervous system inhibitory ability.

4. The system according to claim 3, wherein the integrative analysis based on the physiological mathematical model comprises:
based on the of exercise intensity and heart rate data collected during the aforementioned digital walking experiment, wherein n_{act}(t), HR_{walking}(t), and t={t_{walkstart-1}, t_{walkstop}}], evaluate a regulatory ability of sympathetic nerve on heart rate increase driven by motion, which is represented by AₛfₛC_{NE}, by calculating the cardiac strain response (CR) according to the formula:
$$H {R}_{walking} ( t ) - H {R}_{rest} = H {R}_{Intrinsic} {A}_{s} {f}_{s} ( {n}_{a c t} ( t ) - 1 ) {C}_{N E} \qquad ( 4 )$$
wherein HR_{walking}(t) represents the heart rate varying with time (t) during walking, HRᵣₑₛₜ represents the resting heart rate, and n_{act}(t) represents the exercise intensity varying with time (t) during walking, expressed in metabolic equivalent; and
based on the exercise intensity and heart rate data collected during the aforementioned digital walking test, wherein
[n_{act}(t), HR_{walking}(t), and t={t_{walkstart-1}, t_{walkstop}}] evaluate the heart rate decline rate under the braking effect of the parasympathetic nerve after the cessation of exercise, represented by AₚfₚC_{ACh}, by calculating the cardiac strain inhibition CI according to the formula:
$$d H {R}_{s t o p} ( t ) / dt = {HR}_{Intrinsic} {A}_{p} {C}_{A C h} {f}_{p} ( {n}_{a c t} ( {t}_{s t o p} ) - 1 ) \qquad ( 5 )$$
wherein HRₛₜₒₚ(t) represents the heart rate that changes with time (t) after the cessation of exercise, and tₛₜₒₚ represents the time after the cessation of exercise.

5. The system according to claim 4, wherein the user APP (200) comprises an activity evaluation unit (230) configured to perform the digital walking test and provide personalized exercise training guidance and monitoring, and the personalized exercise training program is generated and recommended by the data service based on the results of multi-vital-sign data integrative analysis based on the data from the digital walking test.

6. The system according to claim 5, wherein the personalized exercise training comprises exercise training guided by a target heart rate, which is used to:
remind the user to increase the intensity of exercise when the exercise heart rate has not reached the lower limit of the target heart rate;
remind the user to reduce the exercise intensity when the exercise heart rate exceeds the upper limit of the target heart rate; and
remind the user to end the exercise training and generate an exercise training report when the exercise duration is reached;
wherein, the upper limit of the target heart rate is the maximum heart rate during the digital walking test, wherein HRᵤₚ=HRₘₐₓ, and the lower limit of the target heart rate is HR_{Low}=HRᵤₚ−CR×1 MET, where CR×1 MET represents the heart rate change value corresponding to an exercise intensity of 1 MET.

7. The system according to claim 6, wherein the specific test comprises a breathing test, the breathing test lasting a total of 9 minutes and including three stages, wherein, in the first stage, the user is guided to breathe freely for 3 minutes in a resting state; in the second stage, the user is guided to breathe rhythmically at a rate of 9 breaths per minute for 3 minutes, and in the third stage, the user is guided to breathe rhythmically at a rate of 6 breaths per minute for 3 minutes, and wherein the multi-vital-sign data integrative analysis comprises integrative analysis based on the physiological mathematical model of heart rate and PPG data of the user acquired by the at least one miniature wearable vital sign acquisition device during the breathing test, to obtain vagal tone VA and stress factor SF.

8. The system according to claim 7, wherein the integrative analysis based on the physiological mathematical model comprises:
estimating respiratory rate based on the PPG data acquired during the breathing test; and
solving the autonomic neurocardiovascular regulation equation based on the provided respiratory rate and heart rate data to obtain the vagal tone VA represented by AₚfₚC_{ACh}, wherein:
$$H R ( t ) = H {R}_{Intrinsic} ( 1 + {A}_{s} {f}_{s} ( {n}_{stress} , {n}_{inf l} , {n}_{others} ) {C}_{N E} + {A}_{p} {f}_{p} ( {n}_{respir} ( t ) ) {C}_{A C h} ) \qquad ( 6 )$$
applying band-pass filtering to the heart rate variations acquired during each stage of the breathing test based on the specified breathing frequency, wherein the autonomic neurocardiovascular regulation equation (6) is rewritten as:
$$d H {R}_{respir} ( t ) / dt = {A}_{p} {C}_{A C h} d {f}_{p} ( {n}_{respir} ( t ) ) / dt \qquad ( 7 )$$
where in the left side of formula (7) represents the heart rate changes caused by breathing during the breathing test, while the right sidenᵣₑₛₚᵢᵣ(t) represents the respiratory signal, and the vagal tone VA=AₚfₚC_{ACh} can be obtained by optimally solving equation (7); and
after solving the respiratory-induced heart rate variability in equation (6), removing the respiratory-induced heart rate variability component to obtain the remaining AC spectrum in the frequency spectrum analysis of the heart rate variability sequence, wherein the stress factor SF related to psychological stress, inflammation, and other internal and external factors is:
$$S F = \{ \sum [ {P}_{H R} ( f ) - {P}_{HR - respir} ( f ) ] \} / \sum {P}_{H R} ( f ) \qquad ( 8 )$$
wherein the P_{HR}(f) is the Fourier transform domain power spectrum of the heart rate sequence, and the P_{HR-respir}(f) represents the Fourier transform domain power spectrum of heart rate changes resulting from the regulation of neural afferent through the vagus nerve during respiration.

9. The system according to claim 8, wherein the self-regulation score for each stage of the breathing test is calculated based on the values of vagal tone (VA) and stress factor (SF) for that stage using the following formula:
$$self - regulation score = [ \frac{V A}{normal value} + ( 1 - S F ) ] / 2 \times 100 \qquad ( 9 )$$
and the overall self-regulation score is the average of the scores from the three stages of the breathing test.

10. The system according to claim 7, wherein the user APP (200) comprises a self-regulation and evaluation unit (240) configured to perform the breathing test and provide personalized rhythmic deep breathing training guidance and monitoring, and the personalized rhythmic deep breathing training program is generated and recommended by the data service based on the results of multi-vital-sign data integrative analysis based on the data from the breathing test.
